(19) United States
(12) Patent Application Publication
(10) Pub. No.: US 2006/0094734 A1
(43) Pub. Date: May 4, 2006

(54) Composition and method for inducing alertness

(76) Inventor: Arnold L. Newman, Bethesda, MD (US)

Correspondence Address: Arnold L. Newman, 8220 Hamilton Spring Court, Bethesda, MD, 20817-2714, US

(21) Appl. No.: 10/981,311

(22) Filed: Nov. 4, 2004

(51) Int. Cl. A61K 31/522; A61K 31/185

(52) U.S. Cl. 514/263.31; 514/553

(57) ABSTRACT

An alertness inducing composition contains the active ingredients caffeine and taurine and various inert substances in a dry formulation. Caffeine and taurine are delivered in an oral formulation that obviates the need for ingesting significant quantities of liquid or sugar.

### CROSS-REFERENCE TO RELATED APPLICATIONS

Not applicable

### FEDERALLY SPONSORED RESEARCH

Not applicable

### SEQUENCE LISTING OR PROGRAM

Not applicable

### BACKGROUND OF THE INVENTION

1. Field of the Invention

This invention relates to a composition and method for increasing the alertness and arousal of a human being. More specifically, it relates to the oral administration of a dry formulation mixture of the amino acid, taurine, and the stimulant, caffeine, to accomplish the synergistic benefits of these components in combination to increase a person's arousal level. Generally, it provides a convenient, measured, and efficient way to increase arousal without having to imbibe significant amounts of liquid or ingest unnecessary sugar or other carbohydrates, or other active ingredients.

2. Related Art

Caffeine has been used as a stimulant and anti-sleep aid for centuries. While the most common source of caffeine is coffee, it is also found in other natural plant sources such as tea, cola nuts, and mate. Chemically, caffeine is 1,3,7-trimethylxanthine, and related to theophylline (1,3-dimethylxanthine) and theobromine (3,7-dimethylxanthine). It is a bitter, white, water-soluble alkaloid powder.

Drinking coffee has long been recognized as a way to increase wakefulness and alertness. Many people drink coffee for its caffeine content to become fully awake and alert in the morning. In many work places, coffee is provided throughout the day so that people can operate at peak alertness and efficiency. Students have long appreciated the benefits of coffee to help them study long into the night. An 8 ounce cup of coffee typically contains between 80 mg and 135 mg or more of caffeine. An equal volume of tea delivers between 30 mg and 70 mg of caffeine.

Caffeine acts as a diuretic and has a stimulatory effect on the central nervous system, the heart, and the respiratory system. Thus, in addition to being a stimulant of the central nervous system, it also has peripheral effects, which, at high doses, can be a problem for some individuals. For example, even moderate amounts of caffeine can cause a rapid heartbeat or palpitations (ectopic heartbeats) in some sensitive people. Other side effects of excessive caffeine can include anxiety, insomnia, diarrhea, diuresis, facial flushing, restlessness, irritability, and trembling. There is a need for a way to achieve the benefits of caffeine while diminishing the possibility of suffering physiologic discomfort.

Currently, caffeine is available as an “over-the-counter” drug in the form of capsules and tablets. For example, the Bristol-Myers Squibb Company markets caffeine as an alertness aid under the brand name No Doz®. This has caffeine as the active ingredient, available in a variety of doses (the maximum dose is 200 mg per caplet), but also includes inactive ingredients well known in the art. GlaxoSmithKline markets caffeine as an alertness aid under the brand name Vivarin®, available in 200 mg tablets. It also includes inactive ingredients well known in the art. Although these brands are described as being as safe as coffee, there is no active ingredient listed that counteracts potential discomfort resulting from excessive caffeine ingestion. There is a need for such an ingredient.

Taurine (2-aminoethanesulfonic acid) is a conditionally essential amino acid because it is not incorporated into proteins, but it is found in a free form in many tissues, particularly muscle and nerve tissue. It is water soluble, and available as a fine crystalline powder. It is a neuroinhibitory transmitter and it may help regulate heart and skeletal muscle contractions, osmotic balance, energy levels, and brain neurotransmitter levels. In rats, the LD 50 of taurine is greater than 5,000 mg/kg.

Taurine appears to have several potentially useful psychological/neurological effects. It has been described as a possible anxiolytic (Chen, SW. et al., Life Sciences 2004 Aug. 6;75(12):1503-11) and an anti-epileptic (El Idrissi A, et al., Adv Exp Med Biol. 2003;526:515-25). It may alleviate visual fatigue (Zhang M, et al. Amino Acids 2004 February;26(1):59-63), attenuate amnesia (Vohra BP and Hui X, Neural Plast. 2000;7(4):245-59), and reduce peripheral sympathetic activity (Chahine R, et al., Arzneimittelforschung. 1994 February;44(2):126-8; Mizushima S, et al., Adv Exp Med Biol. 1996;403:615-22). As a reducer of peripheral sympathetic activity, taurine may counteract the unpleasant effects of caffeine without inhibiting the beneficial stimulating effects of caffeine. Thus, for example, in the “Opinion of the Scientific Committee on Food on Additional information on “energy” drinks” (expressed on 5 Mar. 2003), the view was expressed that “taurine might reduce the cardiovascular effects of caffeine.” (European Commission Health & Consumer Protection Directorate, Brussels Belgium, p. 8, available on the internet at: europa.eu.int/comm./food/fs/sc/scf/out169_en.pdf).

Taurine is marketed in the U.S. by a number of companies. For example, Twin Laboratories of American Fork, Utah, under its Twinlab® brand, markets Taurine as “Mega Taurine Caps,” which are nutrient capsules containing 1000 mg of taurine. Whole Foods Market of Austin, Tex. markets a “vegetarian” taurine as a dietary supplement available in 500 mg capsules.

In the last several years, so-called “energy drinks” have become available in the U.S. market. These soft drinks, usually available in 250 ml quantities, are a mix of ingredients usually including at least one stimulant and additional nutrient components such as amino acids, vitamins, and sweeteners. Caffeine is the most common stimulant in these drinks, but other ingredients, such as taurine, glucuronolactone, guarana, ginseng extract, herbal extracts, and vitamins, can also be found in various “energy drinks.” A popular “energy drink” is Red Bull®, which lists as its ingredients: carbonated water, sucrose, glucose, sodium citrate, taurine, glucuronolactone, caffeine, inositol, niacin, D-pantothenol, pyridoxine HCL, vitamin B12, artificial flavors, colors. Red Bull® Sugar Free drink lists as its ingredients: carbonated water, sodium citrate, taurine, glucuronolactone, caffeine, acesulfame k, aspartame, inositol, xanthan gum, niacinamide, calcium pantothenate, pyridoxine HCl, vitamin B12, artificial flavors, colors. Both versions of Red Bulls contain approximately 1000 mg of taurine and approximately 80 mg of caffeine (within the range of the amount of caffeine in a cup of coffee).

Much of the market success of energy drinks is due to the aura that has been associated with their use by young people in clubs and bars. Despite having a taste that some perceive to be unpleasant and/or artificial, these drinks have acquired a reputation for providing a “legal high” or mild euphoria, and, as a result, have become extremely popular. While the putative feeling of euphoria is likely a myth, enjoyment of these drinks is probably enhanced both by the fun environment in which they tend to be consumed, and the frequent concurrent use of these drinks with alcohol. Although these drinks are expensive, people are willing to buy them because by doing so, they buy into the aura and elan associated with these drinks. Currently, a single can of Red Bull retails for approximately two dollars. This is a lot of money to pay for a drink that delivers 80 mg of caffeine and 1000 mg of taurine. There is a need for a less expensive product that simply delivers caffeine and taurine together, and which is distinct from the psychosocial aura and elan associated with energy drinks.

There appears to be a psychopharmacologic synergy among the ingredients of these drinks. The caffeine is clearly a stimulant, but its effects, in concert with taurine, are different from what one experiences from caffeine alone, such as consuming a single cup of coffee (one can of Red Bull® provides 80 mg of caffeine, which is less than most cups of coffee). Some studies suggest that taurine mitigates adverse effects such as those caffeine may produce.

U.S. Pat. No. 6,261,589 discloses a soft drink that is a nutrient dietary supplement with a psychoactive effect. It is a carbonated beverage containing phenylalanine, vitamin B-6, vitamin C, copper, folic acid, taurine, vitamin B-5 (or pro-vitamin B-5), choline, fruit sugar, caffeine, and optionally green tea. This combination of ingredients is disclosed as a means of increasing energy level and general awareness. The inventors claim that taurine helps prevent excessive sensitivity to noradrenaline and that it promotes “a mellow mood without sedation or tranquilization.” Thus, taurine may help one to avoid the discomfort associated with excessive sensitivity to, or intake of, caffeine. The disclosed soft drink also includes additional ingredients such as vitamins, sugar, and other nutrients.

In Netherlands Patent No. NL1021051C, the inventors disclose a confectionery product based on sugar and/or glucose syrup that includes caffeine and taurine as additives. Although this invention discloses the use of caffeine and taurine for stimulating the central nervous system, respiration, and heart, it is a candy or sweet preparation, and depends upon sugar and/or glucose syrup as important ingredients. In PCT Application No. WO00/62812, the inventor discloses a nutritional composition for improved cognitive performance that comprises caffeine, choline, gamma aminobutyric acid, L-phenylalanine, and taurine in amounts sufficient to improve cognitive performance. In addition to the caffeine and taurine of this invention, several additional ingredients are used beyond what would be needed in a simple alertness aid.

Health-Tech, Inc. of Totowa, N.J., markets “Health-Tech Energy Strips,” which are thin edible strips containing caffeine, taurine, and other ingredients that dissolve easily on the tongue. Due to size limitations, these novelty strips, sold 24 to package, can only deliver a small amount of active ingredients. Three strips are recommended as providing an initially effective dose followed by 1 or 2 strips taken as needed. The consumer is cautioned not to take more than 24 strips during a 24 hour period. This product is fun to use, but not a serious way to self-administer caffeine and taurine precisely, in target amounts.

Despite the known combination of caffeine, and taurine with other active ingredients in energy drinks and sweet products, it is curious that no simple, “over-the-counter” dry formulation pharmaceutical product is available for delivering caffeine and taurine orally. The emergence of energy drinks with caffeine and taurine may have led to the development of candies, sweets and other functional foods that also contain caffeine and taurine. This has not, however, led to the combination of caffeine and taurine in a tablet, capsule, caplet or related formulation. Pharmaceutical and nutriceutical manufacturers have failed to appreciate the advantages of combining caffeine and taurine in a tablet, capsule, caplet or similar formulation.

Accordingly, it is desirable to provide a composition for improving alertness that relies on a combination of ingredients delivered in precise, sufficient amount to operate physiologically to stimulate a person's conscious state without unnecessary discomfort.

### SUMMARY OF THE INVENTION

It is, therefore, an object of the present invention to provide a composition for improving alertness and generally stimulating a person's conscious state, relying on active ingredients that act together to provide a safe, yet higher level of arousal, while minimizing potential discomfort due to untoward side effects.

It is another object of the present invention to provide a composition for improving alertness comprising caffeine and taurine in amounts sufficient to result in a user feeling more awake, alert, responsive and less fatigued, yet more comfortable than would result from using caffeine without taurine.

A further object of the present invention is to provide a method of using the composition for precisely administering caffeine in a measured, dry form as a physiologic stimulant yet with a diminished likelihood of the user suffering discomfort due to the caffeine side effects.

An additional object of the present invention is provide a composition for improving alertness comprising caffeine and taurine without relying on sugar or sweetness to influence product use.

According to the present invention, the above and other objects are accomplished with a composition and method for causing a higher level of alertness, wakefulness, or arousal in a human being by providing caffeine and taurine combined in a capsule, tablet, pill or other dry form for oral administration in a precisely measured amount.

### DRAWINGS

Not Applicable

### DETAILED DESCRIPTION OF THE INVENTION

Caffeine has long been known in the art as a stimulant useful for increasing a person's level of alertness, wakefulness, or arousal. Because it is found in coffee, tea, cocoa, and other foodstuffs, there is a long history of its use and a thorough understanding of its effects and actions. Unfortunately, caffeine is a broadly acting, non-specific stimulant because it stimulates both the central nervous system and the peripheral nervous system and thus has many physiological effects that can be uncomfortable to the user. For example, it can increase heart rate, diuresis, anxiety, and restlessness.

Most people receive caffeine by drinking coffee or tea. This means of caffeine administration delivers inconsistent dosages of caffeine either because of differing concentrations of caffeine or different volumes consumed. For example, in brewing coffee there are many uncontrolled variables such as the coffee bean used, the brewing method followed, and the volume consumed. Furthermore, no coffee is available for administering caffeine along with a substance such as taurine that will mitigate the potential side effects of excessive caffeine ingestions.

Taurine is a non-essential amino acid that has been found to ameliorate some of the uncomfortable effects of caffeine. When caffeine and taurine are taken together, some side effects of caffeine are diminished and the user feels less discomfort, while still benefiting from an increase in alertness.

Although caffeine and taurine are now available in so-called “energy-drinks,” candy, and “energy-strips,” there is no taurine available in over-the-counter caffeine products such as No Doz® or Vivarin®. These products are serious and effective, they deliver measured doses of caffeine, and they do not rely on sweeteners and hype for marketing. They are not novelty items, but they lack taurine.

The present invention combines caffeine and taurine in an easily ingestible dry form such as a capsule, tablet, or pill. Other dry forms such as caplets, powders, or granules are also contemplated. More specifically, any pharmaceutically acceptable, dry dosage form is contemplated by the invention. Examples of such dosage forms include, without limitation, compressed tablets, film coated tablets, chewable tablets, quick dissolve tablets, effervescent tablets, tablets, multi-layer tablets, bi-layer tablets, capsules, soft gelatin capsules, hard gelatin capsules, caplets, lozenges, chewable lozenges, beads, powders, granules, dispersible granules. The present invention also can be incorporated into a chewing gum as a means of delivery. The preparation of any of the above dosage forms is well known in the art. A user may wish to use water or another liquid to aid in swallowing and ingestion of the present invention.

It is possible, in the composition of the present invention, for the dosage form to combine various forms of release, which include, without limitation, immediate release, extended release, pulse release, variable release, controlled release, timed release, sustained release, delayed release, long acting, and combinations thereof. Obtaining immediate release, extended release, pulse release, variable release, controlled release, timed release, sustained release, delayed release, long acting characteristics and combinations thereof is performed using well known procedures and techniques available to one with ordinary skill in the art. None of these particular techniques or procedures constitutes an inventive aspect of this invention.

When caffeine and taurine are incorporated into a candy, sweet, or “energy strip,” there is a possible tendency for the user to ingest too much. The user may perceive the product to be sweet, tasty, and/or fun, but these are not desirable perceptions because they may foster inappropriate and potentially harmful use of the active ingredients. This could be a particular problem for children, who might ingest too much of a candy, sweet, soft drink, or “energy-strip” that, along with other ingredients, contains caffeine and taurine. In contrast, the present invention, delivers caffeine and taurine as a capsule, tablet, pill or other familiar oral medicinal vehicle. This will, in no way, be confused as a candy, sweet, or novelty item. Because the present invention is not a candy or sweet, children will not likely be attracted or drawn to the product and, should a child happen to ingest one dose of the caffeine and taurine product, there will be no desire or incentive to take more.

In the present invention, caffeine and taurine may be ground and mixed together by conventional mixing equipment. The resulting powdered mixture may then be pressed into tablets or placed in gelatin capsules or formulated in another way for oral administration. The product may also contain one or more organic or inorganic additives such as conventional fillers, extenders and excipients. For example, the product may include, but not be limited to, fillers such as lactose or sucrose, mannitol or sorbitol, cellulose preparations and/or calcium phosphates, such as tricalcium phosphate or calcium hydrogen phosphate, binders such as starches, (e.g., maize starch, wheat starch, rice starch, potato starch) gelatin, tragacanth, methyl cellulose, hydroxypropylmethylcellulose, sodium carboxymethylcellulose, and/or polyvinyl pyrrolidone. Flow regulating agents and/or lubricants such as silica, talc, calcium stearate, magnesium stearate and/or polyethylene glycol may be added. Stabilizers known in the art also may be used. Disintegrating agents may be added such as the above-mentioned starches and also carboxymethyl-starch, cross-linked polyvinyl pyrrolidone, agar, or alginic acid or a salt thereof, such as sodium alginate.

It is contemplated that the present invention can be provided as an oral preparation including but not limited to forms such as a tablet to be swallowed whole, a chewable tablet, a quick dissolved tablet, and effervescent tablet, a hard gelatin capsule, a soft gelatin capsule, a caplet or other well known oral formulation. Although the present invention is intended to be in a dry form, it can be taken with water or another liquid to facilitate swallowing and ingestion.

It is not the intent of this invention to deliver nutrients, such as vitamins and minerals, etc., in addition to the two listed active ingredients. Doing so would be unnecessary and contribute nothing to the specific aims of the invention.

The present invention delivers consistent, measured doses of caffeine and taurine. It is provided as a “serious” preparation such as a tablet, capsule, pill, and other forms mentioned herein above. By serious, the inventor means that the invention does not rely on sweetness such as candy preparations and energy drinks, psychosocial marketing “hype,” or novelty formulations such as “energy-strips” to be useful and marketable.

It is contemplated that, in the preferred embodiment, the caffeine content of a single dose formulation of the present invention is about 200 mg or less. It is further contemplated that, in the preferred embodiment, a single dose formulation of the present invention includes an amount of taurine of about 1000 mg or less. Thus, the unit dosage form of the preferred embodiment of the present invention includes about 200 mg or less of caffeine and 1000 mg or less of taurine. An alternative embodiment of the invention is contemplated to contain between about 50 mg to about 200 mg of caffeine and between about 200 mg to about 2000 mg of taurine.

The preferred embodiment and various modifications of the concept underlying the present invention have been set forth. Various other embodiments of the present invention, and modifications of the embodiments herein shown and described will occur to those skilled in the art upon becoming familiar with the disclosure herein. It is to be understood, therefore, that the invention may be practiced otherwise than as specifically set forth herein.

I claim:

1. A dry composition for inducing alertness in a person comprising the active ingredients caffeine and taurine, wherein said dry composition is formulated as a dosage form for oral administration.

2. The composition of claim 1 wherein the ingredients are provided in unit dosage form selected from one of the following: pills, tablets, capsules, caplets, powders, granules, or chewing gums.

3. The composition of claim 1 wherein the ingredients are prepared by employing one or more organic or inorganic additives.

4. The composition of claim 3 wherein said one or more organic or inorganic additives is chosen from the following: an excipient, a preservative, a stabilizer, a suspending agent, a dispensing agent, a diluent or a base wax.

5. The composition of claim 1 wherein an amount of caffeine is about 200 mg or less.

6. The composition of claim 1 wherein an amount of taurine is about 1000 mg or less.

7. The composition of claim 1 wherein the amount of caffeine is from about 50 mg to about 200 mg.

8. The composition of claim 1 wherein the amount of taurine is from about 250 mg to about 2000 mg.

9. A composition for inducing alertness in a person comprising taurine, in the amount of from about 200 mg to about 2000 mg, and caffeine, in the amount of from about 50 mg to about 200 mg, wherein said composition is a dry, oral formulation.

10. A method of inducing alertness in a human being, comprising orally administering a dry, single dose formulation containing caffeine and taurine as the active ingredients.
